(19) United States
(12) Patent Application Publication
(10) Pub. No.: US 2014/0222048 A1
(43) Pub. Date: Aug. 7, 2014

(54) DEBULKING CATHETER

(71) Applicant: Covidien LP, Mansfield, MA (US)

(72) Inventor: Bryan Ladd, Minneapolis, MN (US)

(73) Assignee: Covidien LP, Mansfield, MA (US)

(21) Appl. No.: 14/102,069

(22) Filed: Dec. 10, 2013

Related U.S. Application Data
(60) Provisional application No. 61/736,188, filed on Dec. 12, 2012.

(51) Int. Cl. A61B 17/3207

(52) U.S. Cl. CPC A61B 17/320758
USPC 606/159

(57) ABSTRACT

A catheter for debulking and removing tissue from a body lumen includes an elongate catheter body configured for insertion into the body lumen. The catheter body has opposite distal and proximal end portions, and a longitudinal axis extending between the distal and proximal end portions. A debulking assembly for debulking tissue in the body lumen includes concentric outer and inner cutters disposed generally at the distal end of the catheter body. A drive shaft is operatively connected to at least one of the outer and inner cutters for rotating the at least one of the cutters relative to the other cutter about a rotational axis. Each of the outer and inner cutters includes at least one cutting edge at a distal end of the cutter. Rotation of the at least one of the cutters relative to the other cutter creates a shearing action between the respective cutting edges of the cutters to debulk the tissue in the body lumen.

### CROSS-REFERENCE TO RELATED APPLICATION

This application claims the benefit of priority under 35 U.S.C. §119 to U.S. Patent Application No. 61/736,188, titled DEBULKING CATHETER, which was filed on Dec. 12, 2012, and which is incorporated herein by reference in its entirety for all purposes.

### BACKGROUND

The present invention generally relates to a debulking catheter for removing tissue from a body lumen.

Vascular disease frequently arises from the accumulation of atheromatous material on the inner walls of vascular lumens, particularly arterial lumens of the peripheral and other vasculature, especially peripheral arteries, resulting in a condition known as atherosclerosis. Atherosclerosis occurs naturally as a result of aging, but may also be aggravated by factors such as diet, hypertension, heredity, vascular injury, and the like. Atheromatous deposits can have widely varying properties, with some deposits being relatively soft and others being fibrous and/or calcified. In the latter case, the deposits are frequently referred to as plaque.

Vascular disease can be treated in a variety of ways, including drugs, bypass surgery, and a variety of catheter-based approaches, including those which rely on intravascular debulking or removal of the atheromatous or other material occluding a blood vessel. A variety of methods for cutting or dislodging material and removing such material from the blood vessel have been proposed, generally being referred to as atherectomy procedures. Atherectomy catheters intended to cut or excise material from the blood vessel lumen may employ a rotatable cutting blade (or other tissue-removing element) which can be advanced into or past the occlusive material in order to cut and separate such material from the blood vessel lumen.

It is desirous to provide catheters which can access small, tortuous regions of body lumens and which can remove tissue and/or other occluding materials from within body lumens in a controlled fashion. In one instance, it may be desired to provide atherectomy catheters which can facilitate capturing atheromatous materials. The catheters and methods are for use in a variety of body lumens, including but not limited to coronary, peripheral, and other arteries, and other body lumens.

### SUMMARY

In one aspect, a catheter for debulking and removing tissue from a body lumen includes an elongate catheter body configured for insertion into the body lumen. The catheter body has opposite distal and proximal end portions, and a longitudinal axis extending between the distal and proximal end portions. A debulking assembly for debulking tissue in the body lumen includes concentric outer and inner cutters disposed generally at the distal end of the catheter body. A drive shaft is operatively connected to at least one of the outer and inner cutters for rotating the at least one of the cutters relative to the other cutter about a rotational axis. Each of the outer and inner cutters includes at least one cutting edge at a distal end of the cutter. Rotation of the at least one of the cutters relative to the other cutter creates a shearing action between the respective cutting edges of the cutters to debulk the tissue in the body lumen.

In another aspect, a method for debulking and removing tissue from a body lumen using a catheter including an elongate catheter body configured for insertion into the body lumen and a debulking assembly including concentric outer and inner cutters for debulking tissue in the body lumen includes rotating at least one of the cutters relative to the other cutter with a drive shaft operatively connected to said at least one of the cutters. Rotating the at least one of the cutters creates a shearing action between respective cutting edges of the cutters to debulk the tissue in the body lumen.

Other objects and features will be in part apparent and in part pointed out hereinafter.

### BRIEF DESCRIPTION OF THE DRAWINGS

FIG. 1 is a perspective of a debulking catheter;

FIG. 2 is an exploded perspective of a distal end portion of the catheter;

FIG. 3 is a fragmentary section of the catheter taken through line 3-3 in FIG. 1;

FIG. 4 is a front view of FIG. 1;

FIGS. 5A and 5B are a schematics of a cutting action of the debulking catheter;

FIG. 6 is a section similar to FIG. 3 showing a second embodiment of a debulking catheter; and

FIG. 7 is a section showing a third embodiment of a debulking catheter.

Corresponding reference characters indicate corresponding parts throughout the drawings.

### DETAILED DESCRIPTION OF THE DRAWINGS

Referring now to the drawings, multiple embodiments of a debulking catheter that removes tissue from a body lumen wall are disclosed. In particular, embodiments of the catheter are suitable for use with atherectomy catheters for removing (i.e., excising) an atheroma (i.e., plaque) from an arterial wall, including removing plaque due to in-stent restenosis. The disclosed embodiments, however, may also be suitable for treating stenoses of other body lumens and other hyperplastic and neoplastic conditions in other body lumens, such as the ureter, the biliary duct, respiratory passages, the pancreatic duct, the lymphatic duct, and the like. Neoplastic cell growth will often occur as a result of a tumor surrounding and intruding into a body lumen. Debulking of such material can thus be beneficial to maintain patency of the body lumen. While the remaining discussion is directed toward components of atherectomy catheters for debulking and passing through atheromatous or thrombotic occlusive material in an artery, it will be appreciated that the components may be employed with other types of debulking catheters for removing and/or passing through a variety of occlusive, stenotic, or hyperplastic material in a variety of body lumens.

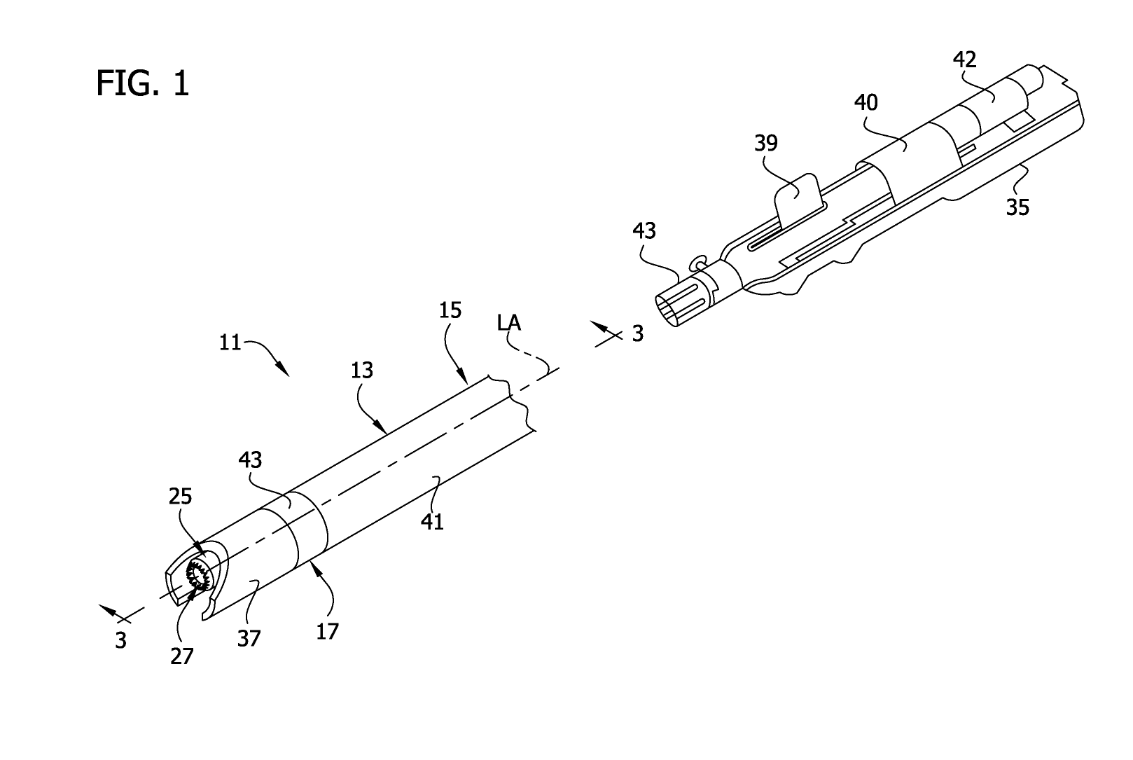

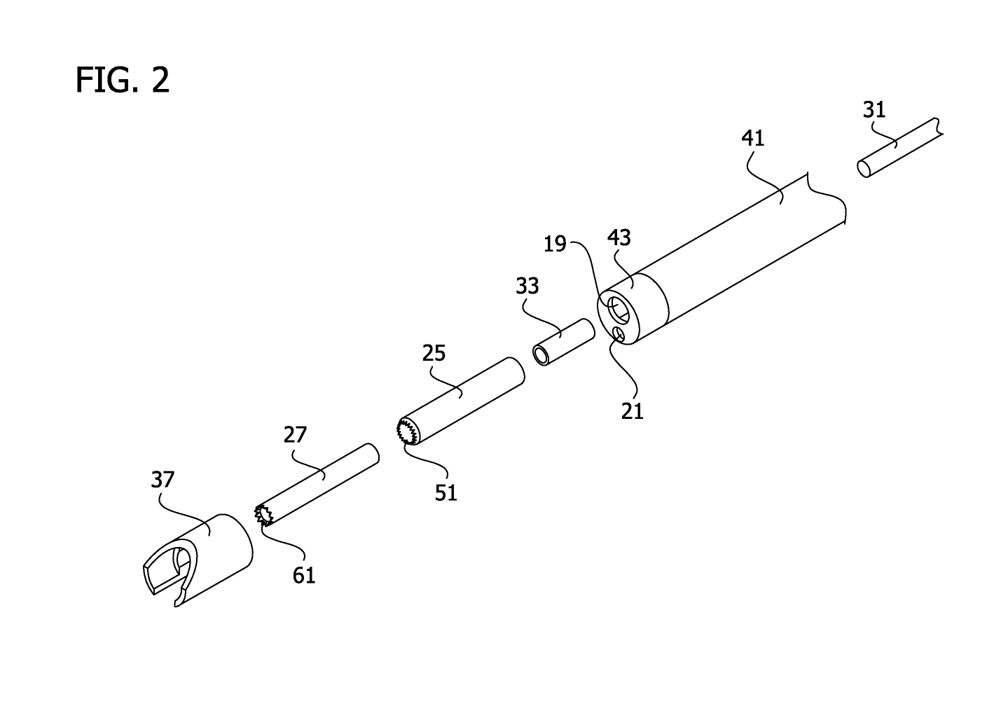

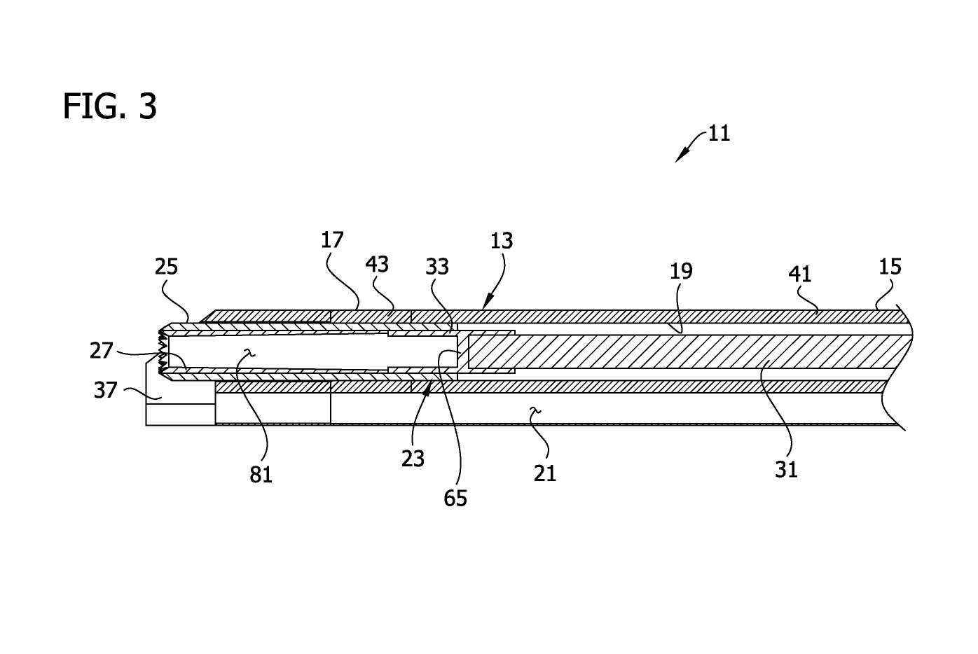

Referring now to FIGS. 1-4, a first non-limiting embodiment of a suitable atherectomy catheter is generally indicated at 11. The catheter comprises an elongate tubular catheter body 13 having a longitudinal axis LA, a proximal end portion 15 and a distal end portion 17. The majority of the catheter body 13, including the proximal end portion 17, may be generally flexible to permit the catheter body 13 to bend and flex facilitating insertion and movement of the catheter 11 in the body lumens of a patient. A primary lumen 19 extends axially through the body 13. The body 13 also defines a guidewire lumen 21 for receiving a guidewire (not shown) to facilitate insertion of the catheter 11 into the target vessel, as is generally known in the art.

A debulking assembly 23 extends through the primary lumen 19. The debulking assembly 23 comprises an outer cutter, generally indicated at 25, and an inner cutter, generally indicated at 27, each of which is positioned generally at the distal end portion 17 of the catheter body 13. The inner cutter 27 extends generally coaxially within the outer cutter 25, and, in the illustrated embodiment, the outer cutter is fixed relative to the catheter body 13 (i.e., the outer cutter is not rotatable relative to the catheter body), while the inner cutter is rotatable about its axis relative to the catheter body and the outer cutter. In one example, the outer cutter 25 is fastened to the inner surface of the primary lumen 19, such as by adhesive, welding, or by using mechanical fasteners. The distal end portion 17 of the catheter body 13 may be generally rigid to provide support for the cutters 25, 27 and to help stabilize the distal end portion during operation of the catheter 11.

A drive shaft 31 is disposed inside the primary lumen 19 and is operatively connected to the inner cutter 27, such as by bearing coupling 33, to drive rotation of the inner cutter, The bearing coupling 33 also couples the inner cutter 27 to the outer cutter 25 to allow for rotation of the inner cutter relative to the outer cutter. As explained below, the drive shaft 31 is connected to a schematically illustrated control handle 35 at the proximal end portion 15 of the catheter body 13. Rotation of the drive shaft 31 causes the inner cutter 27 to rotate about an axis that is generally parallel to the longitudinal axis LA of the catheter body 13.

A cutter housing 37 (forming part of the catheter body 13) is mounted at the distal end portion 17 of the catheter body 13 and at least partially surrounds distal ends of the cutters 25, 27. The cutter housing 37 is beveled or chamfered at its distal end to expose distal circumferential portions of the outer and inner cutters 25, 27. Alternatively, the entire distal circumferential portions of the outer and inner cutters 25, 27 may be exposed or selectively exposable to cut tissue. As will be explained in greater detail below, rotation of the inner cutter 27 relative to the outer cutter 25 creates a scissor-like cutting or shearing action between the cutters to cut and debulk target tissue in a body lumen. It is understood that in other embodiments, the outer cutter 25 may be rotatable relative to the inner cutter 27 (i.e., the inner cutter may be stationary relative to the catheter body 13).

As will be understood by those skilled in the art, the control handle 35 contains electronic controls and indicators for operating the catheter 11. The control handle 35 can include a housing (shown with a cover removed in FIG. 1) that is releasably connectable to the proximal end of the catheter 11 and sized and shaped to be held in a hand of the user. An electric motor 40 (e.g., a DC motor) is contained in the housing, along with a power source 42 (e.g., a battery or other source of DC power) electrically connected to the motor for powering the motor. The handle 35 may also house a microswitch (not shown) for activating the motor 40, and a catheter connector 43 for use in connecting the motor to the proximal end of the drive shaft 31 for driving rotation of the drive shaft and the inner cutter 27. In some embodiments, the motor 40 can rotate drive shaft 31 between 1,000 rpm and 10,000 rpm or more, if desired, in either a clockwise or counterclockwise direction. An input device 39 on an exterior of the control 35 allows the user to control operations of the catheter 11. The inner cutter 27 may also be axially movable along the longitudinal axis LA of the catheter body 13 by an actuator (not shown). The proximal end portion 15 of the catheter body 13 can also be coupled to the distal end portion 17 with a connection assembly (not shown) to allow pivoting or deflection of the distal end relative to the proximal end to selectively expose and cover the cutters 25, 27 without departing from the scope of the disclosure.

The outer cutter 25 comprises an elongate tubular body including teeth or serrations 51 spaced apart from one another on a distal end of the tubular body. In the illustrated embodiment, the serrations 51 are arranged annularly around the tubular body and formed as part of the tubular body. The serrations 51 can be formed separately from the tubular body of the outer cutter 25 and attached to the tubular body without departing from the scope of the disclosure. An outer surface of each serration 51 tapers longitudinally toward a center of the tubular body.

The inner cutter member 27 comprises an elongate tubular body including serrations 61 on a distal end of the tube. In the illustrated embodiment, the serrations 61 are arranged annularly around the tubular body and formed as part of the tubular body. The serrations 61 can be formed separately from the tubular body of the inner cutter 27 and attached to the tubular body without departing from the scope of the disclosure. An inner surface of each serration 61 tapers longitudinally toward the serrations 51 on the outer cutter 25. The taper of the inner surfaces of the serrations 51 on the outer cutter 25, and the taper of the outer surfaces of the serrations 61 on the inner cutter 27 form pointed tips at distal ends of the serrations to facilitate penetration of the cutters through the tissue. Any number of teeth on each of the outer and inner cutters 25, 27 may be employed. The distal ends of the inner and outer cutters 25, 27 need not be serrated.

A proximal end of the inner cutter 27 is mounted on a distal end of the bearing coupling 33. The bearing coupling 33 comprises a tubular body including a divider wall 65. A proximally facing surface of the wall 65 forms a shaft receptacle for receiving a distal end of the drive shaft 31. The bearing coupling 33 is fixed to the drive shaft 31 and the inner cutter 27 such that the drive shaft rotates the inner cutter via the bearing coupling. There is a small clearance between the outer surface of the inner cutter 27 and the inner surface of the outer cutter 25 allowing the inner cutter to rotate relative to the outer cutter, while allowing the respective serrations 51, 61 to create the scissor-like cutting action as the inner cutter rotates. The inner cutter 27 can be connected to the drive shaft 31 in other ways without departing from the scope of the disclosure. For instance, the inner cutter 27 can be attached directly to the drive shaft 31 or attached to the coupling bearing 33 in other ways.

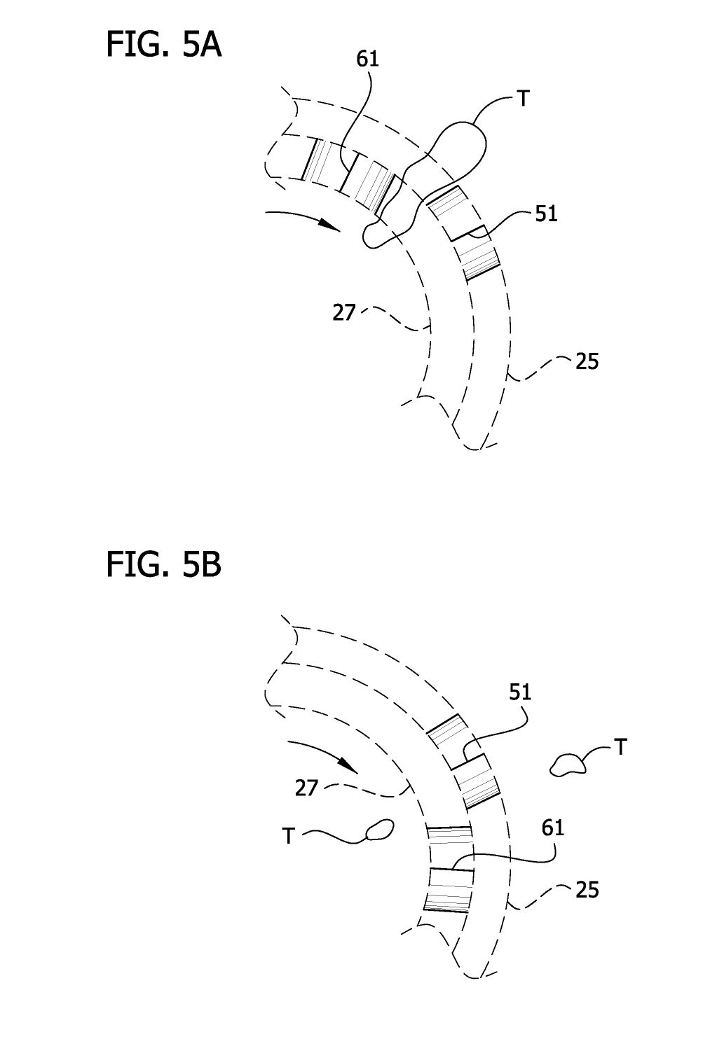

Activation of the input device 39 on the control handle 35 rotates the drive shaft 31 which rotates the inner cutter 27. When the catheter 11 is moved to engage the cutters 25, 27 with target tissue T in the body lumen, the pointed tips of the serrations 51, 61 allow the cutters to penetrate the target tissue. Rotation of the serrations 61 on the inner cutter 27 past the serrations 51 on the outer cutter 25 produces a scissor-like cutting or shearing action to debulk the tissue (FIGS. 5A and 5B). The debulked tissue may be collected in an interior space 81 defined by the inner cutter 27. Although not shown, the catheter 11 may include a transportation mechanism (such as an aspiration device or other means) in communication with the interior space 81 for moving the debulked tissue proximally within the catheter body 13. In one example, the interior space 81 of the inner cutter 27 may be sized to allow multiple cuts to be collected before the catheter 11 has to be removed from the body lumen. When the interior space 81 is full, or at the user's discretion, the catheter 11 can be removed, emptied and reinserted for additional debulking.

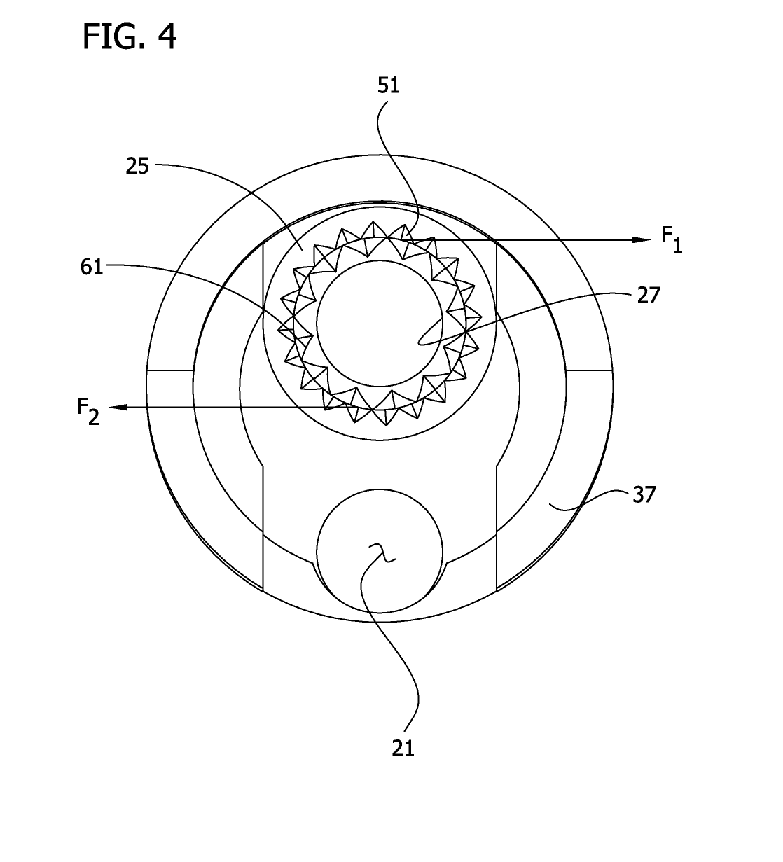

The scissor-like cutting action between the serrations 51, 61 of the cutters 25, 27 provides a laterally balanced cutting force which prevents the distal end portion 17 of the catheter body 13 from swaying from side-to-side during the cutting operation. Conventional atherectomy catheters that use a single side cutter to slice through tissue can generate an unbalanced cutting force which can cause the catheters to sway from side-to-side as the cutter rotates. Because the inner cutter 27 rotates to cut the target tissue and the serrations 51 are uniformly spaced around the inner cutter, the inner cutter generates equal and opposite forces in all opposing tangential directions so that the net tangential force on the distal end portion 17 of the catheter body 13 is minimal. Forces F₁ and F₂ are shown as representative tangential forces which are equal and opposite (FIG. 4). F₁ represents a tangential force generated at a top of the inner cutter 27 and F₂ represents a tangential force generated at a bottom of the inner cutter.

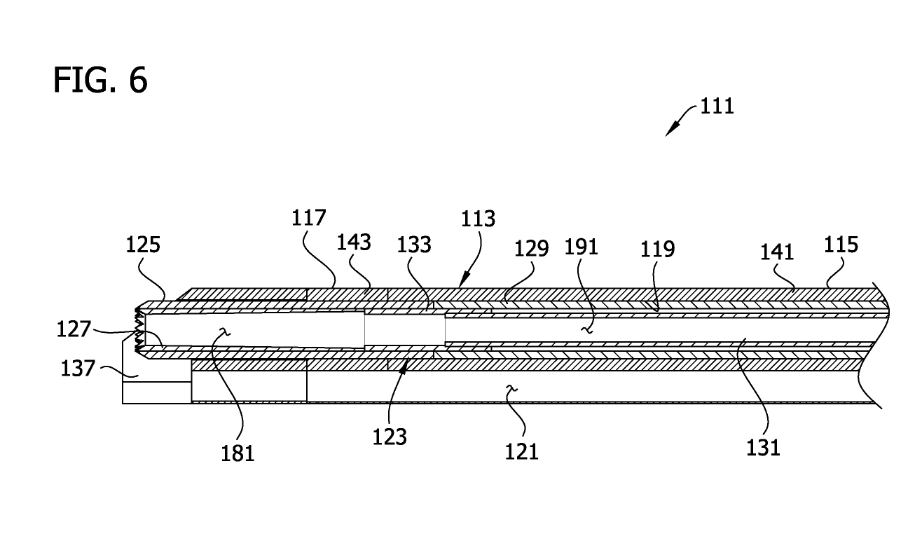

Referring to FIG. 6, a second embodiment of a debulking catheter is indicated generally at 111. The catheter of the second embodiment is similar to the catheter 11 of the first embodiment except a hollow drive shaft 131 is incorporated such that a passage 191 is formed through debulking assembly 123 for removal of the debulked tissue from catheter 111 through the passage. In this construction, bearing coupling 133 may not have a divider wall so as to provide passage through the bearing coupling 133 and into drive shaft 131.

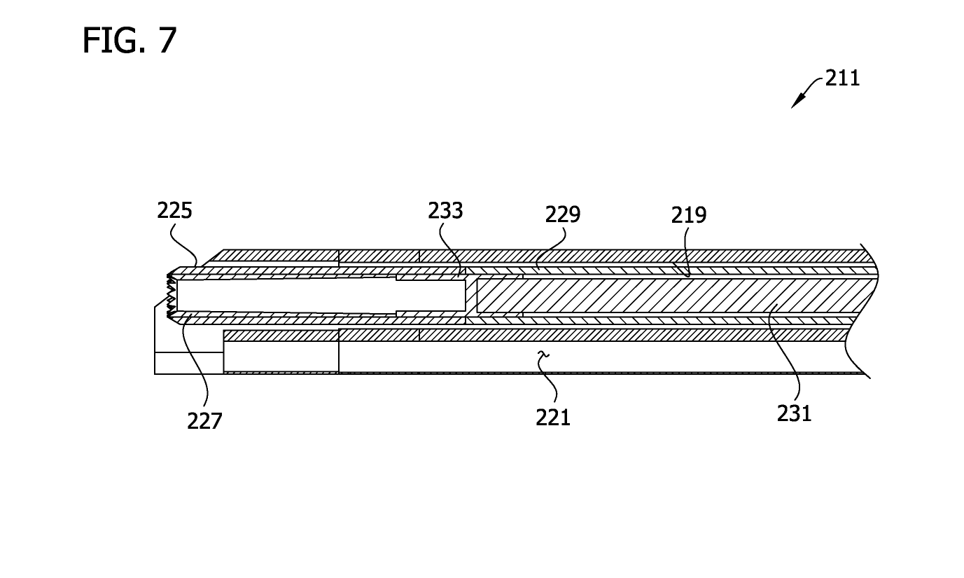

Referring to FIG. 7, a third embodiment of a debulking catheter is indicated generally at 211. The catheter of the third embodiment is similar to the catheter 11 of the first embodiment except the outer cutter 225 is rotatably driven by a drive shaft 229. The drive shaft 229 is configure for coupling to a control handle similar to the control handle 35 illustrated in the first embodiment. The drive shaft 229 comprises a hollow cylindrical member sized to receive bearing coupling 233 and drive shaft 231 associated with inner cutter 227. The drive shaft 231 may be hollow or solid as indicated in the first embodiment. Thus, both the outer cutter 225 and the inner cutter 227 are rotatable relative to catheter body 213. However, the inner cutter 227 may be stationary within the scope of the present disclosure. The control handle can operate to rotate the outer cutter 225 in a first rotational direction (i.e., clockwise) and the inner cutter 227 in an opposite rotational direction (i.e., counterclockwise). The control handle may incorporate a single motor (as illustrated in the handle 35 in the first embodiment) or multiple motors to generate rotation of the respective drive shafts 229, 231. In the instance where a single motor is used, gears may be used to independently rotate the inner and outer cutters 227, 225. And in the instance where multiple motors are used, the handle may have a first motor for rotating the inner cutter 227 and a second motor for rotating the outer cutter 225. Thus, like the catheter 11 in the first embodiment, the relative rotation of the cutters 225, 227 causes a scissor-like cutting action providing balanced cutting forces for cutting and debulking tissue in the body lumen.

A number of embodiments have been described. Nevertheless, it will be understood that various modifications may be made without departing from the spirit and scope of the disclosure. Accordingly, other embodiments are within the scope of the following claims.

What is claimed is:

1. A catheter for debulking and removing tissue from a body lumen comprising:
an elongate catheter body configured for insertion into the body lumen, the catheter body having opposite distal and proximal end portions, and a longitudinal axis extending between the distal and proximal end portions; and
a debulking assembly for debulking tissue in the body lumen comprising concentric outer and inner cutters disposed generally at the distal end of the catheter body, and a drive shaft operatively connected to at least one of the outer and inner cutters for rotating said at least one of the cutters relative to the other cutter about a rotational axis,
wherein each of the outer and inner cutters includes at least one cutting edge at a distal end of the cutter, whereby rotation of said at least one of the cutters relative to the other cutter creates a shearing action between the respective cutting edges of the cutters to debulk the tissue in the body lumen.

2. The catheter set forth in claim 1, wherein the cutting edges of the inner and outer cutters comprise teeth.

3. The catheter set forth in claim 2, wherein the teeth of the respective inner and outer cutters are arranged annularly to define serrated distal ends of the inner and outer cutters.

4. The catheter set forth in claim 3, wherein the teeth of the inner cutter have radially inner and outer surfaces relative to the rotational axis of said at least one of the cutters, and wherein the teeth of the outer cutter have radially inner and outer surfaces relative to the rotational axis of said at least one of the cutters,
the outer surfaces of the teeth of the outer cutter extend radially inward relative to the rotational axis of said at least one cutter and toward the teeth of the inner cutter, and the inner surfaces of the teeth of the inner cutter extend radially outward relative to the rotational axis of said at least one cutter and toward the teeth of the outer cutter.

5. The catheter set forth in claim 1, wherein each of the inner and outer cutters is hollow.

6. The catheter set forth in claim 5, wherein each of the inner and outer cutters are generally tubular, the inner cutter being received within the outer cutter.

7. The catheter set forth in claim 5, wherein the drive shaft is hollow.

8. The catheter set forth in claim 7, further comprising a bearing coupling connecting the drive shaft to a proximal end of the inner cutter, the bearing coupling being hollow.

9. The catheter set forth in claim 1, wherein the outer cutter is fixed against rotation relative to the catheter body, and wherein the drive shaft is connected to the inner cutter so that the inner cutter rotates relative to the outer cutter.

10. The catheter set forth in claim 9, further comprising a bearing coupling connecting the drive shaft to a proximal end of the inner cutter.

11. The catheter set forth in claim 9 wherein the drive shaft is hollow.

12. The catheter set forth in claim 11 further comprising a bearing coupling connecting the drive shaft to a proximal end of the inner cutter, the bearing coupling being hollow.

13. The catheter set forth in claim 1 wherein the drive shaft is connected to the outer cutter so that the outer cutter rotates relative to the inner cutter.

14. The catheter set forth in claim 13 wherein the drive shaft comprises a first drive shaft, the catheter further comprising a second drive shaft operatively connected to the inner cutter, the first drive shaft rotating the outer cutter in a first rotational direction and the second drive shaft rotating the inner cutter in a second rotational direction opposite the first rotational direction.

15. The catheter set forth in claim 14 wherein the first drive shaft is hollow such that at least a portion of the second drive shaft is received inside the first drive shaft.

16. The catheter set forth in claim 1 wherein the outer and inner cutters each comprise elongate serrated tubular cutters.

17. The catheter set forth in claim 16 further comprising plural teeth at the distal end of each of the inner and outer cutters, wherein the teeth of the inner cutter are uniformly spaced around the inner cutter.

18. A method for debulking and removing tissue from a body lumen using a catheter including an elongate catheter body configured for insertion into the body lumen and a debulking assembly including concentric outer and inner cutters for debulking tissue in the body lumen, the method comprising:
rotating at least one of the cutters relative to the other cutter with a drive shaft operatively connected to said at least one of the cutters creating a shearing action between respective cutting edges of the cutters to debulk the tissue in the body lumen.

19. The method set forth in claim 18 wherein the inner cutter is rotated relative to the outer cutter.

20. The method set forth in claim 19 further comprising rotating both the inner and outer cutters in opposite rotational directions.
